(19) United States
(12) Patent Application Publication
(10) Pub. No.: US 2025/0270600 A1
(43) Pub. Date: Aug. 28, 2025

(54) PROCESS FOR PRODUCING FREE FATTY ACIDS

(71) Applicant: Novozymes A/S, Bagsvaerd (DK)

(72) Inventors: Rasmus Boeg HANSEN, Frederiksberg (DK); Jon Martin PERSSON, Bjaerred (SE); Hans Christian HOLM, Hellerup (DK); Kim BORCH, Birkeroed (DK)

(73) Assignee: Novozymes A/S, Bagsvaerd (DK)

(21) Appl. No.: 18/857,409

(22) PCT Filed: Apr. 19, 2023
(86) PCT No.: PCT/EP2023/060141

§ 371 Date: Oct. 16, 2024

(30) Foreign Application Priority Data
Apr. 20, 2022 (DK) PA 2022 00358

(51) Int. Cl. C12P 7/6418; C11C 1/04; C12N 9/20

(52) U.S. Cl. CPC C12P 7/6418; C11C 1/045; C12N 9/20; C12Y 301/01003

(57) ABSTRACT

The present invention relates to a process for producing of free fatty acids comprising a) hydrolyzing fatty acid feedstock with lipase and water in an amount sufficient to produce partial splitting of the fatty acid feedstock in a reactor; and b) mixing said partially split fatty acid mixture in a thermal splitter column under conditions of temperature and pressure effective to substantially complete the splitting of the fatty acid feedstock into free fatty acid and glycerol as by-product.

### FIELD OF THE INVENTION

The present invention relates to a process for producing fatty acids. The present invention further relates to a process for producing free fatty acids comprising hydrolysis of fatty acid feedstock with one or more lipolytic enzymes in the presence of water.

### BACKGROUND OF THE INVENTION

Fatty acids are carboxylic acids having varying degrees of unsaturation and molecular weight. Fatty acids are used in a wide variety of products, such as in soaps and surfactants, lubricants, paints and coatings, candles, and in a variety of other agricultural, industrial, and personal care products.

Though fatty acids and glycerol have been produced synthetically, a substantial portion of these materials are obtained from naturally derived fats and oils. Fats and oils are also known as triglycerides, which are the reaction products of an alcohol, glycerol, and an acid, the fatty acids discussed above. To produce fatty acids and glycerol from fats and oils, the fat or oil is hydrolyzed or “split”, typically by the action of heat and pressure in the presence of water, to break the bonds between the acid and the alcohol.

Typically, the fat or oil is split commercially in a pressure splitter wherein preferably the fat or oil is introduced at one end and water introduced at the opposite end thereof in a countercurrent flow pattern. For smaller volumes of oil, a batch type splitter can also be employed, where high pressure steam provides both water, heat and pressure. In operation, the pressure splitter provides substantial amounts of heat and pressure to the mixture of triglyceride and water to affect the hydrolysis. However, because the triglyceride is hydrophobic, the amount of actual contact between the water phase and the fat phase is relatively low. After a period of time in the splitter individual triglyceride molecules incompletely hydrolyze, splitting off one acid molecule to create a di-glyceride or two acid molecules to form a monoglyceride. The mono- and di-glycerides are less hydrophobic than the starting triglyceride and mix more thoroughly with water. As a result, the mono- and di-glycerides function as emulsifiers to improve mixing of the triglyceride with water. Under the turbulent conditions within the pressure splitter, it is believed that the mono- and di-glycerides improve the extent of mixing between the triglyceride and water, thereby improving the rate of the hydrolysis reaction. Additionally, the formation of free fatty acids (FFA) improves the actual solubility of water in the oil phase significantly, especially because of the high temperature and pressure in the hydrolysis column. And the combination of the emulsification effects of partially split glycerides (monoglycerides (MG), diglycerides (DG)), and the water solubility increasing effect of FFA formation leads to a rapid increase in rate of reaction during the initial reaction stage.

The time period during which the hydrolysis rate is depressed is known as the induction period. During the induction period, the rate of reaction slowly picks up, accelerating until a maximum is reached. Then, towards the end of reaction the rate declines again as substrate is depleted. In a batch type splitter this is very straightforward, but in a continuous column, which runs in a continuous mode, the implications of the induction period are more complicated. The continuous column is tall, in the scale of 50 meters height, and with oil being fed in the bottom and water in the top, the two phases will move in a countercurrent flow. In this case the impact of the induction period on the rate of reaction is best visualized by considering the degree of conversion at different column height points. The slow initial reaction therefore means little conversion is achieved in the lower heights of the column, while majority of the conversion is achieved in a very small part of the height, say, between 25- and 40-meters height. The productivity of the pressure splitter would therefore be increased substantially if the induction period could be eliminated or at least substantially reduced.

It should also be mentioned that a significant byproduct of fat hydrolysis is glycerol. Depending on the choice of process, various glycerol qualities will result. In the uncatalyzed fat hydrolysis at high pressure and temperature described above, glycerol will leave the process as ‘sweetwater’ being typically between 15 and 25 wt % glycerol in water. To obtain the sales-ready commodity, ‘crude glycerin’, holding minimum 85 wt % glycerol a very significant amount of water will therefore have to be evaporated. Therefore, reducing the amount of water needed in the process will be beneficial both economically and environmentally. The glycerol quality might also be impacted by impurities such as degradation products formed due to the high temperature, and removing such impurities is another cost, which adds to the point that reducing the required temperature in the process will bring a benefit in all aspects.

In the Colgate-Emery process, most widely employed, the reaction is conducted in a continuous, counter flow mode, in the absence of a catalyst, at a temperature of 250° C.-330° C. and a pressure of 49-80 Kg/cm² (H. L. Barnebey, The Journal of the American Oil Chemists' Society, Year 1948, pp. 95-99). This process is efficient and vegetable oil/fat splitting yields of 98 percent and above are obtained. However, its applicability to all kinds of vegetable oils and animal fats especially those containing conjugated double bonds and hydroxyl substituents in their fatty acid back bone (castor oil, fish oil etc) is limited. At high temperatures, the damage of fatty acids resulting from oxidation, decomposition, dehydration, polymerization and polycondensation are the usual undesired side reactions. Their propensity increases exponentially with temperature. The by-products formed lead to deterioration in color and odour and to a reduced yield of distillate fatty acid (Russell L. Holliday, Jerry W. King, Gary R. List, Ind. Eng. Chem. Res. Year 1997, Vol. 36, pp. 932-935).

The Twitchell fat splitting process is a batch reaction process. It is not in much use at present. It operates at moderate temperatures (ca. 100 degrees centigrade) and atmospheric pressure employing a homogeneous catalyst. Twitchell reagent comprises of hydrocarbons, oleic acid and concentrated sulphuric acid. This process needs longer contact times (12-24 hrs) than the Colgate-Emery process and fat splitting is 80-85 percent only (L. Hartman, The Journal of the American Oil Chemists' Society, Year 1953, pp. 349-350).

In view of the above, it is desirable to have an efficient, economically beneficial, eco-friendly, catalytic, hydrolysis process of fatty acid feedstock which operates at low water content and produces improved levels of free fatty acids and glycerol as byproduct.

### SUMMARY OF THE INVENTION

The present invention relates to a process for producing of free fatty acids comprising a) hydrolyzing fatty acid feedstock with lipase and water in an amount sufficient to produce partial splitting of the fatty acid feedstock in a reactor; and b) mixing said partially split fatty acid mixture in a thermal splitter column under conditions of temperature and pressure effective to substantially complete the splitting of the fatty acid feedstock into free fatty acid and glycerol.

A general objective of the present invention is to provide an enzymatic oil or fat splitting method which allows for a profitable competitive large-scale process.

This objective is achieved by means of the features of each one of the independent claims. Advantageous further embodiments are defined in the sub-claims. The inventive features provide a short reaction time, an enzyme consumption and a higher glycerol content in the by-product stream.

These and still other objectives and advantages of the present invention will be apparent from the description which follows. In the detailed description below, preferred embodiments of the invention will be described in reference to the accompanying drawings. These embodiments do not represent the full scope of the invention. Rather the invention may be employed in other embodiments. Reference should therefore be made to the claims herein for interpreting the breadth of the invention.

### Definitions

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a step” includes reference to one or more of such steps.

As used herein, “substantial” when used in reference to a quantity or amount of a material, or a specific characteristic thereof, refers to an amount that is sufficient to provide an effect that the material or characteristic was intended to provide. The exact degree of deviation allowable may in some cases depend on the specific context. Similarly, “substantially free of” or the like refers to the lack of an identified element or agent in a composition. Particularly, elements that are identified as being “substantially free of” are either completely absent from the composition or are included only in amounts which are small enough so as to have no deleterious effect on the composition.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight range of about 1 percent to about 20 percent should be interpreted to include not only the explicitly recited concentration limits of 1 percent to about 20 percent, but also to include individual concentrations such as 2 percent, 3 percent, 4 percent, and sub-ranges such as 5 percent to 15 percent, 10 percent to 20 percent, etc.

Fatty acid feedstock: The term “fatty acid feedstock” or “oils or fats” or “vegetable oil feedstock” is defined herein as a substrate comprising triglyceride. In addition to triglyceride, the substrate may comprise diglyceride, monoglyceride, free fatty acid or any combination thereof. In addition, the fatty acid feedstock is any triglyceride stemming from future sources such as fat-producing genetically manipulated microorganisms. In principle, any oils and fats of vegetable or animal origin comprising fatty acids may be used as substrate for producing free fatty acid in the process of the invention. The fatty acid feedstock used according to the present invention may comprise or consist of one or more of algae oil, canola oil, coconut oil, castor oil, coconut oil, copra oil, corn oil, distiller's corn oil, cottonseed oil, flax oil, fish oil, grape seed oil, hemp oil, jatropha oil, jojoba oil, mustard oil, canola oil, palm oil, palm stearin, palm olein, palm kernel oil, peanut oil, rapeseed oil, rice bran oil, safflower oil, soybean oil, sunflower oil, tall oil, oil from halophytes, and/or animal fat, including tallow from pigs, beef and sheep, lard, chicken fat, fish oil, palm oil free fatty acid distillate, soy oil free fatty acid distillate, soap stock fatty acid material, yellow grease, and brown grease or any combination thereof. The fatty acid feedstock may be crude, refined, bleached, deodorized, degummed, or any combination thereof.

Free fatty acids (FFA): A free fatty acid is a carboxylic acid with a long carbon chain. Most naturally occurring fatty acids have an unbranched chain of an even number of carbon atoms, from 4 to 24. Free fatty acids are usually derived from fats (triglycerides (TAG), diglycerides (DAG), monoglyceride (MAG)), phospholipids or lyso-phospholipids. Triglycerides are formed by combining glycerol with three fatty acid molecules. The hydroxyl (HO—) group of glycerol and the carboxyl (—COOH) group of the fatty acid join to form an ester. The glycerol molecule has three hydroxyl (HO—) groups. Each fatty acid has a carboxyl group (—COOH). Diglycerides are formed by combining glycerol with two fatty acid molecules. Monoglycerides are formed by combining glycerol with one fatty acid molecule.

Hydrolysis: The term “hydrolysis” is an enzyme or non-enzyme catalyzed process for production of free fatty acids from glycerides and/or phospholipids by reacting the oil components with H₂O is called hydrolysis process or fat-splitting.

The fat hydrolysis column: Throughout the invention the mentioning such as ‘feeding the fat hydrolysis column’, the main column which further hydrolyses the pre-hydrolysed oil, is meant as adding oil to any existing or future process setup around such hydrolysis hydrolysis column. For example some columns include a deaeration vessel, heat exchanger and/or other unit operations, and such unit operations are considered part of the fat hydrolysis column. The invention should therefore be understood as a way of pretreating the oil in an added process setup that fits into what would otherwise be a simple feed oil line going into what would otherwise only be the fat hydrolysis column setup alone. The expert in the field would quickly realize that such unit operations might be required before the pre splitted oil might enter the main column itself.

### Lipolytic Enzyme

The one or more lipolytic enzyme applied in the method of the present invention is selected from lipases, phospholipases, cutinases, acyltransferases or a mixture of one and more of lipase, phospholipase, cutinase and acyltransferase. The one or more lipolytic enzyme is selected from the enzymes in EC 3.1.1, EC 3.1.4, and EC 2.3. The one or more lipolytic enzyme may also be a mixture of one or more lipases. The one or more lipolytic enzyme may include a lipase and a phospholipase. The one or more lipolytic enzyme includes a lipase of EC 3.1.1.3. The one or more lipolytic enzyme includes a lipase having activity on tri-, di-, and monoglycerides. Lipases: A suitable lipolytic enzyme may be a polypeptide having lipase activity, e.g., one selected from the Candida antarctica lipase A (CALA) as disclosed in WO 88/02775, the C. antarctica lipase B (CALB) as disclosed in WO 88/02775 and shown in SEQ ID NO:1 of WO2008065060, the Thermomyces lanuginosus (previously Humicola lanuginosus) lipase disclosed in EP 258 068), the Thermomyces lanuginosus variants disclosed in WO 2000/60063 or WO 1995/22615, in particular the lipase shown in positions 1-269 of SEQ ID NO: 2 of WO 95/22615, the Hyphozyma sp. lipase (WO 98/018912), and the Rhizomucor miehei lipase (SEQ ID NO:5 in WO 2004/099400), a lipase from P. alcaligenes or P. pseudoalcaligenes (EP 218 272), P. cepacia (EP 331 376), P. glumae, P. stutzeri (GB 1,372,034), P. fluorescens, Pseudomonas sp. strain SD 705 (WO 95/06720 and WO 96/27002), P. wisconsinensis (WO 96/12012); a Bacillus lipase, e.g., from B. subtilis (Dartois et al. (1993), Biochemica et Biophysica Acta, 1131, 253-360), B. stearothermophilus or G. stearothermophilus (JP 64/744992) or B. pumilus (WO 91/16422). Also preferred is a lipase from any of the following organisms: Fusarium oxysporum, Absidia reflexa, Absidia corymbefera, Rhizomucor miehei, Rhizopus delemar (oryzae), Aspergillus niger, Aspergillus tubingensis, Fusarium heterosporum, Aspergillus oryzae, Penicilium camembertii, Aspergillus foetidus, and Thermomyces lanuginosus, such as a lipase selected from any of SEQ ID NOs: 1 to 15 in WO 2004/099400.

A lipase which is useful in relation to the present invention is a lipase having a sequence identity to the mature polypeptide of SEQ ID NO: 2 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or even 100% sequence identity to the polypeptide shown in positions 1-269 of SEQ ID NO: 2 of WO 95/22615 or to the polypeptide shown in SEQ ID NO:1 of WO2008/065060.

Commercial lipase preparations suitable for use in the process of the invention include LIPOZYME® TL 100L, CALLERA™ TRANS and Eversa® Transform, Eversa® Transform 2.0, Novocor AD L (all available from Novozymes A/S), or mixtures thereof.

### Lipase Activity:

In the context of the present invention, the lipolytic activity may be determined as lipase units (LU), using tributyrate as substrate. The method is based on the hydrolysis of tributyrin by the enzyme, and the alkali consumption to keep pH constant during hydrolysis is registered as a function of time

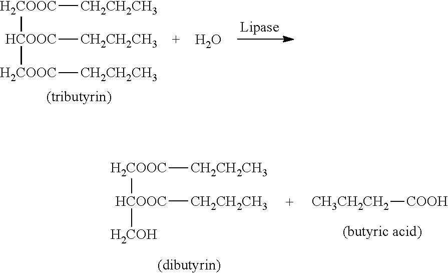

One lipase unit (LU) may be defined as the amount of enzyme which, under standard conditions (i.e. at 30° C.; pH 7.0; with 0.1% (w/v) Gum Arabic as emulsifier and 0.16 M tributyrine as substrate) liberates 1 micromol titrable butyric acid per minute.

Alternatively, lipolytic acitivity may be determined as Long Chain Lipase Units (LCLU) using substrate pNP-Palmitate (C:16) when incubated at pH 8.0, 30° C., the lipase hydrolyzes the ester bond and releases pNP, which is yellow and can be detected at 405 nm.

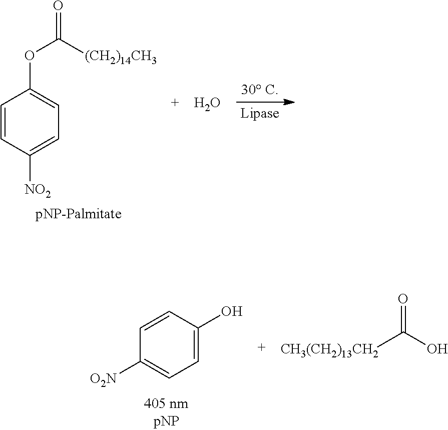

Cutinases: The one or more lipolytic enzyme may include a polypeptide having cutinase activity.

The cutinase may e.g., be selected from the polypeptides disclosed in WO 2001/92502, in particular the Humicola insolens cutinase variants disclosed in Example 2.

Preferably, the one or more lipolytic enzyme is an enzyme having at least 60%, at least 70%, at least 80%, at least 90%, at least 95%, at least 96%, at least 97%, at least 98%, or even at least 99% identity to any of the aforementioned lipases, phospholipases, cutinases, and acyltransferases.

In one embodiment, the one or more lipolytic enzyme has at least 60%, at least 70%, at least 80%, at least 90%, at least 95%, at least 96%, at least 97%, at least 98%, at least or even at least 99% identity to the amino acid sequence shown as positions 1-269 of SEQ ID NO: 2 of WO 95/22615.

Enzyme sources and formulation: The one or more lipolytic enzyme used in the process of the invention may be derived or obtainable from any of the sources mentioned herein. The term “derived” means in this context that the enzyme may have been isolated from an organism where it is present natively, i.e. the identity of the amino acid sequence of the enzyme are identical to a native enzyme. The term “derived” also means that the enzymes may have been produced recombinantly in a host organism, the recombinant produced enzyme having either an identity identical to a native enzyme or having a modified amino acid sequence, e.g., having one or more amino acids which are deleted, inserted and/or substituted, i.e. a recombinantly produced enzyme which is a mutant and/or a fragment of a native amino acid sequence. Within the meaning of a native enzyme are included natural variants. Furthermore, the term “derived” includes enzymes produced synthetically by e.g., peptide synthesis. The term “derived” also encompasses enzymes which have been modified e.g., by glycosylation, phosphorylation etc., whether in vivo or in vitro. The term “obtainable” in this context means that the enzyme has an amino acid sequence identical to a native enzyme. The term encompasses an enzyme that has been isolated from an organism where it is present natively, or one in which it has been expressed recombinantly in the same type of organism or another, or enzymes produced synthetically by e.g., peptide synthesis. With respect to recombinantly produced enzyme the terms “obtainable” and “derived” refers to the identity of the enzyme and not the identity of the host organism in which it is produced recombinantly.

Accordingly, the one or more lipolytic enzyme may be obtained from a microorganism by use of any suitable technique. For instance, an enzyme preparation may be obtained by fermentation of a suitable microorganism and subsequent isolation of an enzyme preparation from the resulting fermented broth or microorganism by methods known in the art. The enzyme may also be obtained by use of recombinant DNA techniques. Such method normally comprises cultivation of a host cell transformed with a recombinant DNA vector comprising a DNA sequence encoding the enzyme in question and the DNA sequence being operationally linked with an appropriate expression signal such that it is capable of expressing the enzyme in a culture medium under conditions permitting the expression of the enzyme and recovering the enzyme from the culture. The DNA sequence may also be incorporated into the genome of the host cell. The DNA sequence may be of genomic, cDNA or synthetic origin or any combinations of these, and may be isolated or synthesized in accordance with methods known in the art.

The one or more lipolytic enzyme may be applied in any suitable formulation, e.g., as lyophilised powder or in aqueous solution.

### DETAILED DESCRIPTION OF THE INVENTION

The present invention relates to a process for producing of free fatty acids comprising a) hydrolyzing fatty acid feedstock with lipase and water in an amount sufficient to produce partial splitting of the fatty acid feedstock in a reactor; and b) mixing said partially split fatty acid mixture in a thermal splitter column under conditions of temperature and pressure effective to substantially complete the splitting of the fatty acid feedstock into free fatty acid and glycerol.

The inventors have found that partial hydrolysis of fatty acid feedstock in presence of a less than molar equivalent amount of water based on fatty acid ester bonds results in surprisingly reduced amount of water after splitting, that the resulting partially split fatty acid mixture can completely be fed into the main splitter column without any limitations of previous methods. Therefore, the pre-splitting step utilizes low amount of water and enzyme which aids in complete splitting of fatty acid feedstock into free fatty acid and glycerol as byproduct.

The claimed process of partial splitting of fatty acid feedstock eliminates the induction period without the attendant disadvantages of previous methods. Specifically, the process employs a partial splitting step wherein a lipase with water is combined with the feedstock to form a reaction mixture. Optionally, water may be already present in the feedstock. The type of water used does not materially affect the reaction. Thus, distilled, tap or deionized water can be used with like effect, while in some cases buffered water might be required such as in cases where a specific enzyme solution requires a defined pH range to properly function. In most such special cases, dilute citric or acetic acid buffer adjusted to the optimum pH would be sufficient.

In one aspect, the process further comprises separation of free fatty acid.

In one aspect, the fatty acid feedstock is defined herein as a substrate comprising any source of fatty acids, including methyl esters, ethyl esters, triglycerides, diglycerides, monoglycerides, or any combination thereof.

In one aspect, the fatty acid feedstock is a naturally derived oil or fat, or a mixture thereof.

In one aspect, the fatty acid feedstock is any triglyceride stemming from future sources such as fat-producing genetically manipulated microorganisms.

In one aspect, the fatty acid feedstock is derived from one or more of algae oil, canola oil, coconut oil, castor oil, coconut oil, copra oil, corn oil, distiller's corn oil, cottonseed oil, flax oil, fish oil, grape seed oil, hemp oil, jatropha oil, jojoba oil, mustard oil, canola oil, palm oil, palm stearin, palm olein, palm kernel oil, peanut oil, rapeseed oil, rice bran oil, safflower oil, soybean oil, sunflower oil, tall oil, oil from halophytes, and/or animal fat, including tallow from pigs, beef and sheep, lard, chicken fat, fish oil, yellow grease, and brown grease or any combination thereof.

The invention in its broader aspects relates to a process of producing free fatty acid from fatty acid feedstock in a reactor comprising the combining in a first step of the fatty acid feedstock with a suitable amount of an effective lipase in the presence of water to partially split the glycerides present in the fatty acid feedstock, and mixing the partially split glycerides present in the fatty acid feedstock in the thermal splitter column under conditions of temperature and pressure effective to substantially complete the splitting of the glycerides present in the fatty acid feedstock into component fatty acids and glycerol, wherein the production of the fatty acid and glycerol from the partially split glyceride present in the fatty acid feedstock is increased relative to a glyceride not treated with the lipase.

In one aspect, the liquid lipase product, based on the specific enzyme protein, is dosed from 1-100 mg/kg of fatty acid feedstock, such as 2.5-60 mg/kg of fatty acid feedstock, such as from 5-40 mg/kg of fatty acid feedstock. Levels of lipase outside this range may be used, as well as different lipase enzymes. The lipase is mixed with water or optionally in buffer solution prior to blending with the feedstock.

In one aspect the lipase is an immobilized lipase on solid particles such as silica or resins. In that case the dosage of immobilized enzyme product, based on weight of oil, may range from 0.5-100%, such as 1-100% or such as 2-100%. The ranges are broad, because such dosage would entirely depend on the design of the system for employing the enzyme. One type of system could, as an example, be a fluidized bed with confinement of a large amount of stationary enzyme with a continuous flow of oil through the bed, in which case the dosage would relatively high. Another type could be a well-mixed reactor holding a smaller dosage of immobilized enzyme, which could be filtered off and reused continuously or batchwise. Those are examples, but should not be seen as limiting, because an expert in the field of employing immobilized enzymes would see how such system could be designed in numerous ways.

In one aspect, the lipase used in step a) is selected from the group consisting of: Aspergillus oryzae lipase; Aspergillus niger lipase; Thermomyces lanuginosa lipase; Candida Antarctica lipase A; Candida Antarctica lipase B; Candida cylindracae lipase; Candida deformans lipase; Candida lipolytica lipase; Candida parapsilosis lipase; Mucor miehei lipase, Candida rugosa lipase; Corynebacterium acnes lipase; Humicola lanuginose lipase, Cryptococcus spp. S-2 lipase; Fusarium culmorum lipase; Fusarium heterosporum lipase; Fusarium oxysporum lipase; Mucor javanicus lipase; Rhizomucor miehei lipase; Rhizomucor delemar lipase; Burkholderia (Pseudomonas) cepacia lipase; Pseudomonas sp, ATCC 21808 lipase, Pseudomonas camembertii lipase; Pseudomonas fluorescens lipase; Rhizopus lipase; Rhizopus arrhizus lipase; Staphylococcus aureus lipase; Geotrichium candidum lipase; Hyphozyma sp. lipase; Klebsiella oxytoca lipase; B. stearothermophilus or G. stearothermophilus lipase and wildtype orthologs and homologs thereof; and variants thereof.

In one aspect, the lipase is of Regio-, and positional specificity/selectivity all relate to the preference of the enzymes towards reacting the 1, 2, and 3 positions of the glycerides.

The preferred lipase is capable of hydrolyzing any glyceride ester bond as quickly as possible on any position and with as little slowdown of reaction speed as possible during the extent of reaction. One may also use a 1,3-position specific enzyme that would indirectly promote acyl-migration, resulting in formation of 1- or 3-monoglyceride from a 2-monoglyceride or similar migration of 1, 2- or 2,3-diglyceride to become 1,3-diglyceride. Acyl migration is not a critical requirement of the invention but will promote the rate beneficially. Lipase regioselectivity is often fluid, although the concept itself is used in a black and white manner, meaning an enzyme described as 1,3 specific will often have a high rate of reaction on the 1- and 3-positions while still being able to react the 2-position, albeit significantly slower.

The invention uses lipases (triacylglycerol lipase), i.e., enzymes that catalyze the hydrolysis of ester bonds in triglycerides (triacylglycerol). They are classified as EC 3.1.1.3 according to Enzyme Nomenclature. The lipases are characterized by their regioselectivity, i.e., the specificity of the lipases towards the acyl groups in the 3 different positions of a triglyceride. Thus, the microbial regioselective (or 1,3-specific) lipase hydrolyzes acyl groups in the 1- and 3-positions with little or no activity in the 2-position, whereas the regionally non-specific lipase hydrolyzes acyl groups in all three positions at comparable rates. The regioselectivity of a lipase may be determined as described in WO8802775, in WO 8901032 or in Example 8 of WO 9414940.

Several enzymes might be used in combination. It is known that specific combinations of enzymes can result in increased net rates of reaction when reacting on glycerides. This is because different enzymes might have specificities regarding fatty acid chain lengths and degrees of unsaturation as well as positions of these different fatty acids on the glycerol molecule. The combination of different enzymes can in some cases then cause a synergistic effect where one enzyme works well on a specific combination of position, fatty acid length and degree of unsaturation, where the other enzyme is weak.

Regio unspecific lipase: The unspecific lipase may be microbial, e.g., fungal or bacterial, particularly but not limited to one derived from the following genera and species as described in the indicated publications: Candida, C. rugosa (also called Diutina rugosa), C. cylindracea, C. antarctica lipase A or B (WO 8802775), Pseudomonas, P. cepacia (WO 8901032), Streptomyces (WO 9414940). It may also be a variant obtained by substitution, deletion or insertion of one or more amino acids in of one of the indicated lipases, e.g., as described in WO 9401541.

Regio selective microbial lipase: The specific microbial lipase may be fungal or bacterial, e.g., derived from the following genera and species as described in the indicated publications: Thermomyces, T. lanuginosus (also known as Humicola lanuginosa, EP 305216, U.S. Pat. No. 5,869,438), Rhizomucor, R. miehei, Fusarium, F. oxysporum (WO 9826057), or a lipase variant, e.g., as described in WO 9707202. The specific microbial lipase may also be a cutinase, i.e., an enzyme which also has cutinase activity (EC 3.1.1.74), e.g. a cutinase from Humicola, H. insolens (WO 9613580) or a cutinase variant, e.g. as described in WO 00/34450 or WO 0192502.

The positionally unspecific or specific lipase may be microbial, e.g. bacterial, archeal or fungal, either filamentous or yeast-like and be derived from culturable or unculturable strains, as well as metagenomic sequences. It may particularly be derived but not limited to the following taxonomic orders, genera and species as exemplified and described in the indicated publications: Psedomonadales: Pseudomonas, P. flourescens (WO2018021324), P. cepacia (WO 8901032); P. aeruginosa; Streptomycetales: Streptomyces, S. griseus (WO2011150157), (WO 9414940); Burkholderiales, Burkholderia; B. cepacia (also called Pseudomonas cepacia) (WO9100908); Streptomycetales: Streptomyces, S. griseus (WO2011150157); Bacillales: Geobacillus thermocatenulatus (WO12077614); Ustilaginales: Moesziomyces, M. antarcticus (also called Candida antarctica) A or B (WO 8802775); Eurotiales; Thermomyces, T. lanuginosus (also known as Humicola lanuginosa, EP 305216, U.S. Pat. No. 5,869,438); Penicillium, P. camemberti (WO2006084470), Aspergillus tubingensis (WO200294123-A2); Evansstolkia, E. leycettana (WO2014147219), Talaromyces, T. thermophilus (WO200266622); Hypocreales: Fusarium, Fusarium sp. (WO2018114938), F. oxysporum (WO9826057), or a lipase variant, e.g. as described in WO9707202, Mucorales: Rhizopus, R. arrhizus (also known as R. oryzae) WO2015181118); Rhizomucor, R. miehei (WO2020014407); Mucor, M. circinelloides (WO2014147127); Absidia A. reflexa (WO2004099400). The microbial lipase may also be a Type-B carboxylesterase, recognized in literature as a particular type within lipase EC 3.1.1.3. This lipase may have an origin such as Saccharomycetales; Geotrichum, G. candidum (WO9401567); Limtongozyma, L. cylindracea (also called Candida cylindracea) WO2019044531); Diutina D. rugosa (also called Candida rugosa) WO2018213482). Eurotiales: Aspergillus, A. niger (WO2004018660) Rasamsonia, R. emersoni (WO2014202616), Sordariales: Chaetomium olivicolor (WO2016090474); Hypocreales: Gibberella zeae (WO2006047469); Magnaporthales: Pyricularia, P. grisea (WO2006047469).

The microbial lipase may also be a cutin hydrolase, i.e. an enzyme which also has cutinase activity (EC 3.1.1.74, Synonyms: cutinase, cutin esterase, PET hydrolase), e.g. a cutinase of bacterial or fungal origin such as Streptosporangiales, Thermobifida, T. fusca; (WO2012099018-A1), Ideonella, I. sakaiensis (WO2021005199); Eurotiales: Aspergillus, A. oryzae (WO2018099965); Magnaporthales: Magnaporthe grisea (WO10/107560); Sordariales: Humicola, H. insolens (WO 9613580) or a cutinase variant, e.g. as described in WO 00/34450 or WO 0192502; Thermothelomyces, T. thermophilus (WO2012027282-A2) Eurotiales: Aspergillus, A. oryzae (WO2018099965-A1, WO2014081884-A1), Evansstolkia, E. leycettana (WO2018099965-A1); Rasamsonia R. emersonii (WO2014202616); Hypocreales: Fusarium, F. solani (WO2014081884); Magnaporthales, Magnaporthe, M. grisea (WO2010107560); Helotiales: Oculimacula yallundae (WO2014059541).

The lipase may further be from a yeast such as Candida, Kluyveromyces, Pichia, Rhodotorula, Saccharomyces, Schizosaccharomyces or Yarrowia; or from a filamentous fungal origin such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Gloeophyllum, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rasamsonia, Rhizomucor, Rhizopus, Schizophyllum, Scytalidium, Talaromyces, Thermoascus, Thielavia, Tolypocladium, Trichoderma, Trichophaea, Verticillium, Volvariella, or Xylaria.

The lipase may also be a variant obtained by substitution, deletion or insertion of one or more amino acids in of one of the indicated lipases, e.g. as described in WO 9401541. The lipase may be non-heterogeneously or heterologous expressed using a microbial expression system.

Immobilized lipase: The lipase may be immobilized, e.g., by covalent linkage with glutaraldehyde to particulate silica, by adsorption on a particulate macroporous weakly basic anion exchange resin, by adsorption on polypropylene or by cross-linking, particularly with glutaraldehyde, e.g. with addition of MgSO₄. The immobilization may be carried out as described in EP 140452, WO 8902916, WO 9005778, WO 9015868, EP 232933 or U.S. Pat. No. 4,665,028. The lipases may be mixed before immobilization, or they may be immobilized separately. In the latter case, the two immobilized lipases may be mixed, or they may be used separately in consecutive steps.

In one aspect, temperature in step a) of process is in the range of about 20° C. to about 120° C., such as 25° C. to about 90° C., such as 30° C. to about 80° C.

In one aspect, the reactor in step a) of the process is a batch or continuous mode.

In one aspect, the reaction time of step a) of the process is from 20 minutes-24 hours, such as 40 minutes-12 hours, such as 1-6 hours in a batch or continuous process.

In one aspect, the reactor is a batch reactor, a plug flow reactor, or a continuous stirred tank reactor (CSTR), when a plurality of reactors are used to react the feedstock with lipase and water, the reactors are arranged in series, in parallel, or in combination of series and parallel.

In addition to the batch process for the above lipase presplitting of triglycerides, it has been found that significant advantages result from carrying out lipase presplitting in a continuous process.

In one aspect, lipase and water in a continuous stirred tank reactor setup is added to one or more of the reactors.

In one aspect, CSTR reactors may be one or more, which run in a series with a separation step in between and/or after the final CSTR reactor before entering the thermal splitter column.

In one aspect, the amount of water added in the reactor is about 0.01-2.0, such as 0.05-1.0, such as 0.1-0.5 molar equivalents based on the fatty acids present in the feedstock (including fatty acids bound in glycerides).

In one aspect, water utilization is at least 70%, such as at least 80%, such as at least 85%, such as at least 90% of water added in step a).

In one aspect, the water concentration after reaction in total reaction mixture of step a) is below 10000 ppm, more preferably below 7500 ppm and most preferably below 5000 ppm.

A continuous lipase presplitting process for triglycerides can be carried out as follows. A triglyceride oil to be treated, is introduced continuously into a reaction vessel at an elevated temperature. A lipase slurry in water is simultaneously introduced on a continuous basis into the reaction vessel. The flow rates of the triglyceride and of the slurry are adjusted to provide water based on the weight of triglyceride, and to provide a residence time for the triglyceride in the reaction vessel, depending on the temperature and on the activity of the lipase used in the process. The mixture in the reaction vessel is thoroughly mixed throughout the process, using any agitation or stirring means that will accomplish such thorough mixing. The effluent presplit triglyceride can then be processed directly in a thermal splitter column.

Optionally, the residual water of hydrolysis, containing both free glycerol and lipase activity, may be recovered by phase separation. This separation can for example may be done external to the presplitting reactor, for example using a centrifuge or under gravity using an auxiliary settling tank. The resulting isolated, depending on separation efficiency, light phase is processed in a thermal splitter column. It is conceivable that some fraction of the heavy sweet water phase can be recycled to the presplitting reactor to further increase the glycerol concentration.

Alternatively, to achieve recycle of residual lipase, the phase separation can be carried out internal to the presplitting reactor by forming a quiescent settling zone inside the presplitting reactor, below the location where presplit effluent is withdrawn from the reactor. Any arrangement having a hydraulic radius sufficiently large such that the terminal settling velocity of the water droplets that coalesce in the quiescent zone exceeds the upward velocity of the presplit fat can be used. An auxiliary effluent exit location is provided for removing the presplit triglycerides from the reactor contents. Any desired recycle ratio can be achieved by balancing the rate that presplit triglycerides are removed from above the settling zone with the rate effluent is withdrawn from the reactor.

Partially split fatty acid mixture is passed in flow to a preheater which preheats the mixture by heat exchanger before entering the column.

The operation of commercial thermal splitter column is well known in the industry and the invention does not aim to change such operation markedly, except for making the columns operable at improved environmentally friendly conditions with product quality kept intact or improved. Essentially, triglyceride in the form of an oil, liquified fat, or a blend thereof is introduced into a thermal splitter column with water, and heat is applied. During startup, as the temperature increases, so does the pressure. The column, once operating in steady state will, as the expert in the field knows, operate at essentially constant conditions with concentration, temperature and pressure gradients within the column itself. The operating with pre-splitted oil will change such gradient markedly, allowing for operation at lower temperature or water dosage, or with a higher throughput through increased flow of oil and thereby productivity. A balance of all three improvements is also obtainable.

In batch splitters, the components are mixed by agitation. In continuous splitters, the triglyceride is typically introduced from the bottom, water from the top, and the difference in densities and the input pumping force causes mixing.

In one aspect, the temperature in step b) of process is in the range of about 180° C. to about 260° C., such as 190° C. to about 250° C., such as 200° C. to about 240° C.

The triglyceride is mixed in the continuous splitter with water, which might be added as liquid water and/or as steam of various pressure, and which in total is dosed from 15 to 80%, preferably from 25-70% and most preferably from 30-65% by weight of the feed oil. Batch pressure splitting involves temperatures in the range of 180° C. to 260° C., and pressures preferably in the range of 10-70 bar. Water content in the batch process is similar to the ranges above.

In one aspect, the pressure in step b) of the process is in the range of about 10-70 bar, such as 15-60 bar, such as 20-50 bar.

In one aspect, the presplitting process may be carried out optionally in presence of buffer, the buffer strength should preferably be sufficient to keep pH within the optimal range throughout the majority of the extent of reaction, where pH decreases due to formation of acidic free fatty acids. The optimal range will be lipase specific, with some lipases showing their highest activity at pH above pH 7.0 and others at lower levels such as pH 4.0. Depending on the fatty acid feedstock the final pH near reaction completion can be as low as pH 3.0, requiring pH control for some enzymes. pH might also be controlled through pH-stat principles, where acid or base such as citric acid or sodium hydroxide is added as reaction progresses.

The resulting free fatty acid are separated by methods known to the art, preferably by distillation.

In one aspect, the split yield of the pre-splitted oil in the column is greater than 85%, such as 90%, such as 95%, such as 98%. The expert in the field will realize that such yield values are largely dependent on the treated feedstock oil. A largely unsaturated oil such as soybean will have a higher tendency to polymerize at the temperatures employed in the column, resulting in a lower yield of intact fatty acids leaving the column than what is achievable with an oil such as the stearin fraction of palm oil.

A significant improvement provided by the invention is the ability to operate the column at reduced temperatures, which will reduce formation of byproducts thereby improving the final yield of the distilled free fatty acid product leaving the entire combined process.

### EXAMPLES

### Example 1: Presplittinq with Crude Palm Oil (CPO) as Oil Substrate

### Reaction Conditions:

30 g crude palm oil with 3.3 wt % FFA measured.
2% water added based on the weight of oil.
0.05% Eversa Transform 2.0 HS added based on the weight of oil.
60 degrees Celsius at 250 rpm mixing in a shaking incubator oven.

### Procedure:

Premix and preheat oil and water. Then add the enzyme and react.

Samples of 2 mL are heated to 99 deg C. for 10 minutes to inactivate the enzyme before spinning the sample to isolate the denatured enzyme in the bottom of the sample, avoiding any continued reaction in the sample.

FFA is measured using the AOCS official method Ca 5a-40.

### Results

25.8 wt % FFA measured in the oil phase after 4 hours.

That is roughly equivalent to 68% conversion of the added water, leaving just 0.64 wt % water in the total mixture.

FFA is within the preferred range above, while the water concentration is a outside the most preferred range.

Longer reaction time, higher temperature, pH adjustment, improved mixing efficiency and increased enzyme dosage would all further improve water conversion.

### Example 2: Presplittinq with Refined Palm Kernel Oil as Oil Substrate

### Reaction Conditions:

30 g refined palm kernel oil.
2% water added based on the weight of oil.
10-0.02% Eversa Transform 2.0 HS added based on the weight of oil.
60 degrees Celsius at 250 rpm mixing in a shaking incubator oven.

### Procedure:

Premix and preheat oil and water. Then add the enzyme and react.

Samples of 2 mL are heated to 99 deg C. for 10 minutes to inactivate the enzyme before spinning the sample to isolate the denatured enzyme in the bottom of the sample, avoiding any continued reaction in the sample.

FFA is measured using the AOCS official method Ca 5a-40.

### Results

| | | Water | Remaining water in |
|---|---|---|---|
| Time | FFA measured | conversion | the total mixture |
| (hours) | (wt % in light phase) | (%) | (wt %) |
| 5 | 22.15 | 86.1 | 0.26 |
| 24 | 24.51 | 95.4 | 0.09 |

The results after 5 hours are all within the preferred levels of the invention.

While there has been shown and described what are at present considered the preferred embodiments of the invention, it will be obvious to those skilled in the art that various changes and modifications can be made therein without departing from the Scope of the invention defined by the appended claims.

1: A process for producing free fatty acids, said process comprising:
a) hydrolyzing a fatty acid feedstock with one or more lipolytic enzymes and water in an amount sufficient to produce a partially split fatty acid mixture of the fatty acid feedstock in a reactor; and
b) mixing said partially split fatty acid mixture in a thermal splitter column under conditions of temperature and pressure effective to substantially complete the splitting of the fatty acid feedstock into free fatty acid and glycerol.

2: The process of claim 1, wherein the process further comprises separation of free fatty acid.

3: The process according to claim 1, wherein the fatty acid feedstock comprises a mixture of triglyceride with monoglyceride and/or diglyceride.

4: The process according to claim 1, wherein the fatty acid feedstock comprises a naturally derived oil or fat, or a mixture thereof.

5: The process according to claim 1, wherein the fatty acid feedstock comprises a triglyceride from a fat-producing genetically manipulated microorganism.

6: The process according to claim 1, wherein the fatty acid feedstock is derived from one or more of microbial oil, algae oil, canola oil, coconut oil, castor oil, coconut oil, copra oil, corn oil, distiller's corn oil, cottonseed oil, flax oil, fish oil, grape seed oil, hemp oil, jatropha oil, jojoba oil, mustard oil, canola oil, palm oil, palm stearin, palm olein, palm kernel oil, peanut oil, rapeseed oil, rice bran oil, safflower oil, soybean oil, sunflower oil, tall oil, oil from halophytes, and/or animal fat, or any combination thereof.

7: The process according to claim 1, wherein said temperature in step a) is in the range of about 20° C. to about 120° C.

8: The process according to claim 1, wherein said temperature in step b) is in the range of about 180° C. to about 260° C.

9: The process according to claim 1, wherein said pressure in step b) is in the range of about 10-70 bar.

10: The process according to claim 1, wherein said one or more lipolytic enzymes is dosed from 1-100 mg/kg of fatty acid feedstock.

11: The process according to claim 1, wherein one or more lipolytic enzymes used in step a) is selected from the group consisting of: Aspergillus lipase; Aspergillus niger lipase; Thermomyces lanuginosa lipase; Candida Antarctica lipase A; Candida Antarctica lipase B; Candida cylindracae lipase; Candida deformans lipase; Candida lipolytica lipase; Candida parapsilosis lipase; Mucor miehei, Candida rugosa lipase; Corynebacterium acnes lipase; Humicola lanuginose, Cryptococcus spp. S-2 lipase; Fusarium culmorum lipase; Fusarium heterosporum lipase; Fusarium oxysporum lipase; Mucorjavanicus lipase; Rhizomucor miehei lipase; Rhizomucor delemar lipase; Burkholderia (Pseudomonas) cepacia lipase; Pseudomonas sp, ATCC 21808, Pseudomonas camembertii lipase; Pseudomonas fluorescens lipase; Rhizopus lipase; Rhizopus arrhizus lipase; Staphylococcus aureus lipase; Geotrichium candidum lipase; Hyphozyma sp. lipase; Klebsiella oxytoca lipase; and wildtype orthologs and homologs thereof; and variants thereof.

12: The process according to claim 1, wherein the reactor in step a) is a batch or continuous mode.

13: The process according to claim 1, wherein the reaction time of step a) is from 20 minutes to 24 hours in a batch or continuous process.

14: The process according to claim 1, wherein the reactor is a batch reactor, a plug flow reactor, or a continuous stirred tank reactor (CSTR), and when a plurality of reactors are used to react the feedstock with one or more lipolytic enzymes and water, the reactors are arranged in series, in parallel, or in combination of series and parallel.

15: The process according to claim 14, wherein one or more lipolytic enzymes and water in a continuous stirred tank reactor is added to one or more reactors.

16: The process according to claim 1, wherein one or more CSTR reactors run is in a series with a separation step in between and/or after the final CSTR reactor before entering the thermal splitter column.

17: The process according to claim 1, wherein the amount of water added in the reactor is about 0.01-2.0 molar equivalents based on the fatty acid present in the feedstock.

18: The process according to claim 1, wherein at least 70% of water added in step a) is utilized.

19: The process according to claim 1, wherein the water concentration after reaction in total reaction mixture of step a) is below 10000 ppm.

20: The process according to claim 1, wherein the split yield is greater than 85%.
